(19) United States
(12) Patent Application Publication
(10) Pub. No.: US 2009/0275990 A1
(43) Pub. Date: Nov. 5, 2009

(54) ANTERIOR CERVICAL PLATE WITH LOCKING MECHANISM

(75) Inventor: Albert Enayati, Los Angeles, CA (US)

Correspondence Address: KRAMER & AMADO, P.C., 1725 DUKE STREET, SUITE 240, ALEXANDRIA, VA, 22314, US

(73) Assignee: RHAUSLER, Inc., Redwood City, CA (US)

(21) Appl. No.: 12/115,368

(22) Filed: May 5, 2008

(51) Int. Cl. A61B 17/70; A61B 17/04

(52) U.S. Cl. 606/291; 606/280; 606/301; 606/305

(57) ABSTRACT

An anterior cervical plating system is disclosed that includes an anterior cervical plate with a locking mechanism feature, bone screws, and a locking element. The bone screws fasten the anterior cervical plate to the spinal vertebrae and locking elements are inserted into a housing to prevent withdrawal of the bone screws from the vertebrae. The anterior cervical plate has at least one pair of adjacent bone screw holes, with the housing mounted perpendicularly between adjacent bone screw holes. When the bone screws are fully inserted into the vertebrae through the anterior cervical plate, the locking element prevents the retraction of the bone screws from the vertebrae. The elastically-deformable locking element holds the bone screws inside the screw holes.

### BACKGROUND OF THE INVENTION

1. Field of the Invention

This invention relates to an anterior cervical plate, and, in particular, it relates to such a plate with a new and improved locking element.

2. Description of the Related Art

The present invention relates to cervical plates used for fusing cervical vertebrae in the treatment of spinal disorders, and, more particularly, to components for locking the fasteners for such plates.

An increasingly accepted procedure for treating spinal disorders involves using substantially rigid plates to hold vertebrae in desired spatial relationships and orientations relative to each other. The upper cervical spine can be approached anteriorly or posteriorly, although anterior approaches are of more interest in connection with this invention. In either case, holes are drilled and tapped in at least two of the vertebrae. These holes then receive screws or other fasteners used to secure the plate.

The holes are accurately positioned with reference to openings formed through the cervical plate. In some cases, the screws may be self-tapping. Typically, the plate is curved about its longitudinal axis to facilitate contiguous surface engagement of the plates with the vertebrae. With the plate maintained against the vertebrae, the fasteners are secure within the holes. As a result, the plate maintains the attached vertebrae in a desired spacing and orientation with respect to each other.

One of the problems associated with this technique is the tendency of the screws or other fasteners to gradually work loose after fixation. Slight shock or vibration of the vertebrae, due to walking, climbing stairs, or more vigorous activity by the patient following treatment increases this tendency, jeopardizing the integrity of fixation. Moreover, as the fasteners work loose, the outward protrusion of the heads over other components of the fasteners can be a source of discomfort and presents the risk of trauma to surrounding soft tissues.

Many cervical plates of the present type are known, each having various arrangements for securing the bone screws. Notwithstanding the development of the art to date, a need exists for improvements in arrangements for holding the bone screws in place after the bone screws have secured the cervical plate onto the adjacent vertebrae.

Anterior cervical plates that are fabricated from bioabsorbable materials such as polylactide offer a number of advantages relative to metallic plates. They do not interfere with techniques such as X-ray imaging or magnetic resonance imaging (MRI). Such plates also possess inherent material flexibility, thereby allowing load sharing with the graft. Because these plates are not metallic, there is no risk of corrosion. Furthermore, when heated, it is easy to shape the plates to match the contours of the vertebrae.

Anterior cervical plates can be used to stabilize, immobilize and align the cervical spine. For example, an intermediate disc or discs can be removed and replaced by a cage, a mesh, a bone graph/plug, or an allograft/autograft. In addition to removal of the discs, the intermediate vertebrae can also be removed. The cervical plate can also be used to correct instability of the cervical spine caused by trauma, tumors, advanced degenerative disc disease, cervical deformities caused by lordosis, kyphosis, or other conditions.

While the above approaches yield favorable results in certain circumstances, there remains a need for greater flexibility in positioning and orienting the bone screws or fasteners, and for a simpler, more reliable means of counteracting the tendency of the bone screws to work loose after cervical plate fixation.

Therefore, there is a need for a cervical plate and fixation system in which the bone screws or other fasteners are more securely retained and less likely to work loose, without the need for auxiliary screws or other additional fixtures. There is a further need for a locking element that has a tracking geometry similar to the outside surface of the screw head and fits snugly into the screw head track, thereby locking the bone screws head inside of the anterior cervical plate screw hole and preventing the retraction of the bone screws from the vertebrae. In addition, there is a need for a system including cervical plates with locking features for retaining fastener heads, in combination with tools for conveniently manipulating the locking features to selectively retain or release the fasteners.

The foregoing objects and advantages of the invention are illustrative of those that can be achieved by the various exemplary embodiments and are not intended to be exhaustive or limiting of the possible advantages which can be realized. Thus, these and other objects and advantages of the various exemplary embodiments will be apparent from the description herein or can be learned from practicing the various exemplary embodiments, both as embodied herein or as modified in view of any variation which may be apparent to those skilled in the art. Accordingly, the present invention resides in the novel methods, arrangements, combinations, and improvements herein shown and described in various exemplary embodiments.

### SUMMARY OF THE INVENTION

In light of the present need for an anterior cervical plate with an improved locking element, a brief summary of various exemplary embodiments is presented. Some simplifications and omissions may be made in the following summary, which is intended to highlight and introduce some aspects of the various exemplary embodiments, but not to limit its scope. Detailed descriptions of preferred exemplary embodiments adequate to allow those of ordinary skill in the art to make and use the inventive concepts will follow in later sections.

In various exemplary embodiments, an anterior cervical plate may stabilize, immobilize, and align vertebrae. This plate has a new and improved arrangement for securing the bone screws in place after the cervical plate has been attached to the vertebrae.

Thus, in various exemplary embodiments, an anterior cervical plate may include an exterior surface, an interior surface, and a number of bone screw holes for attaching the cervical plate to the vertebrae. Specifically, in accordance with the present invention, there is provided at least one pair of adjacent bone screw holes, preferably transversely aligned, wherein the cervical plate has a locking element housing that is perpendicularly mounted between the adjacent bone screw holes and movable between a first, open position, where the locking element is not in contact within the two bone screw holes, thereby allowing insertion or removal of the bone screws, and a second, locking position wherein the locking element is in contact with at least a portion of each of the two bone screw holes, thereby locking those bone screws in place. The locking element of the present invention is intended essentially to prevent the screws from backing out.

In various exemplary embodiments, the locking element may be resilient, ductile, and movable from an open position to a closed position by application of an external force above the elastic limit of the locking element. The locking element may comprise an elastically deformable body that is capable of locking the head of the bone screw inside of the anterior cervical plate's screw hole, thereby preventing retraction of the bone screws from the vertebrae.

In various exemplary embodiments, an elongated locking element may be mounted into the locking element housing of the cervical plate between two adjacent bone screw holes. This locking element is movable to a first position wherein it is completely out of contact with the two adjacent holes so as to permit the insertion of and removal of the bone screws. The locking element is then arranged to be partially in contact with the two bone screw holes when the bone screws are secured in place. The screw head, when maintained in the aperture against the interior surface region, is advantageously contained, so that the head does not protrude outwardly beyond the plate or other structural element.

In various exemplary embodiments, the aperture may be sized with respect to the shank to allow a pivoting of the screw with respect to the structural member about at least one transverse axis. More preferably, the fastener is pivotable about all transverse axes passing through a given point in the first aperture, thereby defining a conical volume within which the fastener is selectively positionable.

In various exemplary embodiments, locking and releasing tools may be provided. A locking tool includes a shaft with a locking end that can be attached to the proximal surface of the locking element. This tool is capable of inserting the locking element into the locking element housing of the cervical plate and allowing the deformation of the locking element body against the screw head into a closed position, thereby preventing the retraction of the bone screws from the vertebrae. A releasing tool, having a shaft with a locking end that can be attached to the proximal surface of the locking element in a closed position, applies a tension force to the proximal surface of the locking element, thereby advancing the locking element out of the housing to an open position. This movement allows withdrawal of the bone screws.

In various exemplary embodiments, a system including the structural member and screws may further include tools for securing and removing the structural member. In particular, the heads of the screws can include non-circular recesses. A drive tool with a drive shaft can be provided. One end of the shaft has a non-circular profile that corresponds to the profile of the recess. The screw shanks, in this approach, are externally threaded, so that rotation of the drive tool turns the screws. The drive tool can incorporate a sleeve that is coaxial with the drive shaft and flexure members that grip the fastener turned by the drive tool.

In various exemplary embodiments, anterior cervical plates may have a virtually limitless number of configurations. Cervical plates are generally referred to by the number of levels that they overlie, wherein the word “level” refers to the number of intervening intervertebral spaces that are spanned. Thus, for example, a three level cervical plate would span the four vertebrae and three intervening intervertebral spaces. Such a plate can be connected by a single central bone screw through a central bone screw hole instead of by two adjacent bone screw holes. In various configurations, an opening will be provided between adjacent vertebrae for viewing the intervening intervertebral space.

Thus, in various exemplary embodiments, cervical plates and other structural members may be secured to vertebrae or other osseous material in a manner that more reliably prevents fasteners from working loose in response to shock or vibration. The locking elements are movable, through either plastic or elastic deformation, to close positions that allow insertion and removal of fasteners, and alternatively are positioned to prevent fasteners from working free of their respective apertures in the cervical plate or other structural members.

### BRIEF DESCRIPTION OF THE DRAWINGS

In order to better understand various exemplary embodiments, reference is made to the accompanying drawings, wherein:

FIG. 1 is a perspective view of an anterior cervical plate with a locking mechanism feature configured to be secured to two vertebrae;

FIG. 2 is a perspective view illustrating the internal components of the locking mechanism of FIG. 1;

FIG. 3 is a front view of a locking element;

FIG. 4 is a front view of a bone screw for the anterior cervical plating system;

FIG. 5 is a perspective view of an anterior cervical plating system with a locking mechanism assembly;

FIG. 6 is a perspective view of another anterior cervical plating system; and

FIG. 7 is a front view of a locking and releasing tool.

### DETAILED DESCRIPTION OF THE PREFERRED EMBODIMENTS OF THE INVENTION

Referring now to the drawings, in which like numerals refer to like components or steps, there are disclosed broad aspects of various exemplary embodiments.

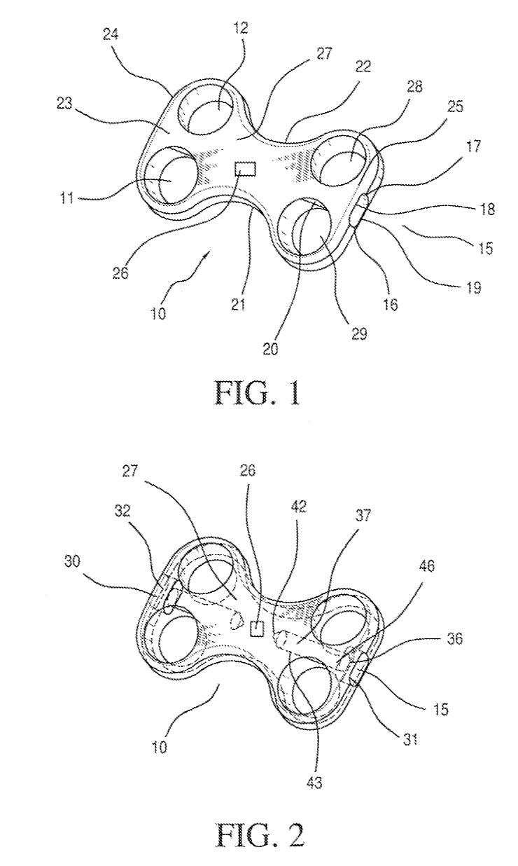

FIG. 1 is a perspective view of an anterior cervical plate 10 with a locking mechanism feature configured to be secured to two vertebrae. Cervical bone plate 10 has a medial side 21, a lateral side 22, an interior surface 23, a distal surface 24, a proximal surface 25, and an exterior surface 27. Locking mechanism 15 is positioned at proximal surface 25. Mechanism 15 has a medial surface 16, a lateral surface 17, an interior surface 18, an exterior surface 19, and an aperture 20. A first pair 11, 12 and a second pair 28, 29 of screw holes are located in plate 10, holes that are intended for use by four bone screws that secure plate 10 to the upper and lower vertebrae. In addition to screw holes 11, 12, 28, 29, cervical plate 10 has at least one simple hole 26, a window for viewing the interior of the intervertebral spaces. There will generally be at least one such window in the vicinity of each intervertebral space.

FIG. 2 is a perspective view illustrating the internal components of the locking mechanism 10 of FIG. 1. Locking mechanisms 15, 30 are positioned on opposite sides, corresponding respectively to the proximal 25 and distal 24 surfaces shown in FIG. 1. Locking mechanisms 15, 30 fit into respective apertures 31, 32. Mechanism 15 has a longitudinal end 36, a central orifice 37, a pointed, distal end 42, and a proximal section 46. Mechanism 15 is adjacent and perpendicular to second pair of screw holes 28, 29, while mechanism 30 is adjacent and perpendicular to first pair of screw holes 11, 12. As will be described further in connection with FIG. 3, a side of mechanism 15 defines a track 43.

FIG. 3 is a front view of a locking element 40. When mounted into each aperture 31, 32 of each locking mechanism 15, 30 of cervical plate 10, locking element 40 blocks withdrawal of bone screws from the vertebrae. Locking element 40 has a trapezoidal, distal end 39 and a rectangular, proximal head 41. A tool engages tool socket 45 in proximal head 41 of locking element 40 to permit either insertion or removal of locking element 40 from each aperture 31, 32. In addition, locking element 40 includes longitudinal engagement tracks 43, 44 that engage matching tracks on bone screws. Locking element 40 also possesses a proximal hole 46, a distal hole 47, and a central hole 49.

Locking element 40 fits into each mechanism 15, 30, and may be mounted perpendicularly between a first pair 11, 12 and a second pair 28, 29 of transversely aligned screw holes. When bone screws are fully inserted into the vertebrae through anterior cervical plate 10, locking element 40 prevents retraction of bone screws from the vertebrae. Locking element 40 is also elastically deformable due to its central hole 49. Locking element 40 may be fabricated from polylactide/polylactic acid (PLA), a copolymer of 70:30 poly L-lactide-co-D, L-lactide, or from other bioabsorbable materials.

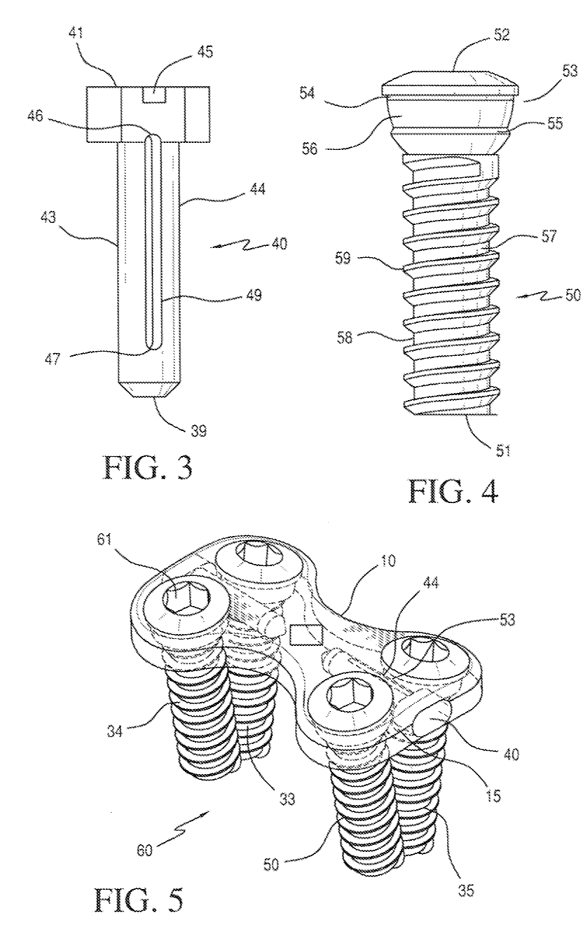

FIG. 4 is a front view of bone screw 50 for the anterior cervical plating system. Bone screw 50 has a distal end 51, a proximal surface 52, and a longitudinal body 57. Screw 50 also has a shank 58 and a helical thread 59. Head portion 53 of bone screw 50 has an upper track 54, a lower track 55, and a central track 56 between tracks 54, 55 that is indented relative to tracks 54, 55. Tracks 54, 55, 56 in head portion 53 of bone screw 50 are contoured to engage corresponding tracks 43, 44 on locking element 40, previously described above in connection with FIG. 3.

When bone screw 50 is fully inserted into the vertebrae through anterior cervical plate 10, locking element 40 is forced to close mechanism 15. Longitudinal engagement tracks 43, 44 of locking element 40 fit snugly at aperture 20 into tracks 54, 55, 56 in head portion 53 of bone screw 50, thereby mechanically locking bone screw 50 inside screw hole 29 and preventing retraction of bone screw 50 from the vertebrae. Screw hole 29, previously described above in connection with FIG. 1, has a shape matching the perimeter of proximal surface 52 of bone screw 50.

FIG. 5 is a perspective view of an anterior cervical plating system 60 with a locking mechanism assembly. Bone screws 33, 34, 35, 50 respectively fit into holes 12, 11, 28, 29, previously shown in FIG. 1. Bone screw 35 resembles bone screw 50, previously described in connection with FIG. 4, but is disposed on the opposite side of locking element 40 and tilted in a mirrored configuration. Locking element 40 secures screws 35 and 50 in position.

A screwdriver, inserted into bone screw socket 61, pushes screws 33, 34, 35, 50 into the vertebrae through screw holes 12, 11, 28, 29 of cervical plate 10. Each screw 33, 34, 35, 50 has an elongated, longitudinal, threaded shank 57 that matches the perimeters of screw holes 11, 12, 28, 29 in plate 10. After an appropriate amount of turning, screws 33, 34, 35, 50 penetrate into the vertebrae to a sufficient depth to engage exterior surface region 27 of plate 10, shown in FIG. 1, with the vertebrae.

When screws 33, 34, 35, 50 are fully inserted into screw holes 11, 12, 28, 29, head portions 53 of screws 33, 34, 35, 50 engage interior surface region 23, thereby fixing the structural member to the vertebrae. Locking element 40 moves from an open position to a closed position, where it engages mechanism 15. Tracks 43, 44 of each locking element 40 fit snugly at aperture 20 into screw tracks 54, 55, 56 of head portion 53, thereby mechanically locking bone screws 33, 34, 35, 50 inside screw holes 11, 12, 28, 29 and preventing the retraction of screws 33, 34, 35, 50 from the vertebrae.

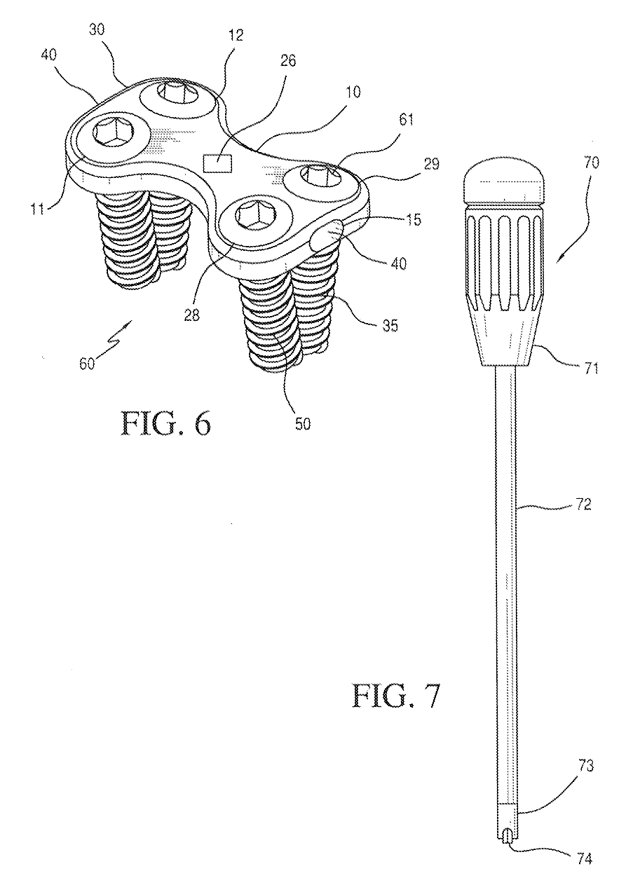

FIG. 6 is another perspective view of anterior cervical plating system 60. In this case, a tool would be first inserted into a bone screw socket 61 on the lower right side instead of a socket on the upper left side. Locking elements 40 would occur on both sides of system 60, each locking element 40 securing one pair of bone screws.

FIG. 7 is a front view of a locking and releasing tool 70. Tool 70 has a handle 71, a shaft 72, an attachment 73, and a locking tip 74. Tip 74 fits snugly into tool socket 45 of locking element 40. Tool 70 can insert locking element 40 into housing 15 in cervical plate 10 and allow deformation of central hole 49 against screw tracks 54, 55, 56. This prevents retraction of bone screws 33, 34, 35, 50 from the vertebrae. In the closed position, shaft 72 attaches to proximal head 41 of locking element 40 and applies tension in order to advance locking element 40 out of housing 15, 30 to an open position to allow withdrawal of associated bone screws 33, 34, 35, 50.

The preferred material for plate 10, bone screws 33, 34, 35, 50, and locking element 40 is polylactide, also known as polylactic acid (PLA). PLA is a copolymer of 70:30 poly L-lactide-co-D, L-lactide that is derived from lactic acid, a chemical that occurs naturally in the human body. The copolymer maintains its strength during the heating process and through hydrolysis slowly breaks down into lactic acid molecules. These molecules are subsequently metabolized into water and carbon dioxide, a gas that is released from the body through the lungs.

Plates made from such materials are easily contourable when heated to the shape of the vertebras. Other materials include polymer blends of glycolide and/or lactide homopolymer, copolymer and/or glycolide/lactide copolymer and polycaprolactone copolymers, and/or copolymers of glycolide, lactide, poly L-lactide-co-DL-lactide, caprolactonc, polyorthoesters, polydioxanone, trimethylene carbonate, and/or polyethylene oxide, or any other bioabsorbable material. Medical grade stainless steel, medical grade titanium, or hybrid construction may be applicable.

Further, the plate made from titanium has sufficient ductility to permit curving the plate about a longitudinal axis so that the cervical plate more readily conforms to the vertebrae. The ductility also plays a role in the use of retaining features that capture each fastener within its aperture, as will be explained. Certain stainless steels are suitable as alternatives to titanium in the plate and fastener construction.

Locking element 40 also may be fabricated from a pseudoelastic shape memory alloy. By way of example, one such pseudoelastic shape memory alloy might be a nickel titanium alloy such as Nitinol, which is available from Flexmedics of Minneapolis, Minn., among others. The use of such a material, in combination with the normal orientation of the barbs relative to the anchor body, permits the barbs to initially deflect inwardly to the extent required to permit the anchor to move forward in the bone tunnel, yet still resiliently “spring back” toward their normal, outwardly projecting position so as to prevent the anchor from withdrawing back out the bone tunnel.

Although the various exemplary embodiments have been described in detail with particular reference to certain exemplary aspects thereof, it should be understood that the invention is capable of other embodiments, and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only, and do not in any way limit the invention, which is defined only by the claims.

What is claimed is:

1. An anterior cervical plating system for fusing vertebrae, comprising:
an anterior cervical plate having at least two screw holes and a locking mechanism;
at least two bone screws disposed within said screw holes;
and a locking element disposed inside said locking mechanism between said screws,
wherein said locking element prevents retraction of said screws from said vertebrae.

2. The system of claim 1, further comprising:
a body having an exterior surface and an interior surface;
a first pair of screw holes disposed at one end of said body;
a second pair of screw holes disposed at another end of said body; and
a window opening disposed between said first and second pairs of holes.

3. The system of claim 2, wherein said locking mechanism is mounted perpendicularly between adjacent holes in each pair of screw holes to accommodate said locking element.

4. The system of claim 1, wherein said locking element further comprises:
a medial surface;
a lateral surface; and
an elongated, longitudinal aperture.

5. The system of claim 4 wherein said longitudinal aperture of said locking element permits elastic deformability of said body.

6. The system of claim 4 wherein said longitudinal aperture is resilient, ductile, and movable from an open position to a closed position by application of an external force that exceeds its elastic limit.

7. The system of claim 5, further comprising an elastically deformable body that can lock heads of said bone screws inside said screw holes and prevent retraction of said bone screws from the vertebrae.

8. The system of claim 4, wherein said medial surface and said lateral surface of said locking element engage an outside surface of a bone screw head and fit snugly into a screw head track, thereby mechanically locking said head of said bone screw inside a screw hole and preventing retraction of said bone screw from said vertebrae.

9. The system of claim 2, wherein each of said bone screws comprises an elongate longitudinal threaded shank and a screw head larger in diameter than said shank, said shank is adapted for insertion through said screw holes of said plate, and said shank penetrates said vertebrae to a depth sufficient to bring said exterior surface into surface engagement with said vertebrae while said screw head engages said interior surface, thereby fixing a structural member substantially integrally to said vertebrae.

10. The system of claim 4, wherein said bone screws have a similar geometry to said medial and said lateral surfaces of said locking element.

11. The system of claim 4, wherein screw head tracks engage said medial and lateral surfaces of said locking element and said locking element elastically deforms to fit snugly into said screw head tracks, thereby preventing withdrawal of said bone screws from said vertebrae.

12. The system of claim 4, further comprising a locking and releasing tool having a shaft with a locking end that can engage a proximal surface of said locking element, wherein said tool can insert said locking element into said locking mechanism of said plate and allow deformation of said locking element against a screw head into a closed position, thereby preventing retraction of said bone screws from said vertebrae.

13. The system of claim 4, wherein application of tension to a proximal surface of said locking element advances said locking element to an open position to permit withdrawal of said bone screws.

14. The system of claim 9, wherein screw holes of said plate are sized with respect to said screw heads to allow pivoting of a screw head with respect to a structural member about all transverse axes passing through a given point within an aperture.

15. The system of claim 1, wherein said locking mechanism, said bone screws, and said locking element are made from a material selected from the group consisting of a bioabsorbable, moldable polymer, a pseudoelastic shape memory alloy, titanium, stainless steel, and a cobalt-chrome alloy.

16. An anterior cervical plating system for fusing vertebrae, comprising:
an anterior cervical plate having at least a pair of screw holes and a locking mechanism;
wherein each screw hole is sized to receive a bone screw, and
wherein said locking mechanism is mounted perpendicularly between adjacent apertures in said pair of screw holes to accommodate a locking element.

17. A locking element for at least one pair of bone screws, comprising:
a body having an exterior surface and an interior surface;
a medial surface;
a lateral surface; and
an elongated, longitudinal aperture,
wherein said longitudinal aperture of said locking element permits elastic deformability of said body.

18. The locking element of claim 17, wherein said longitudinal aperture is resilient, ductile, and movable from an open position to a closed position by application of an external force that exceeds its elastic limit.

19. The locking element of claim 17, wherein said medial surface and said lateral surface of said locking element engage an outside surface of a bone screw head and fit snugly into a screw head track, thereby mechanically locking said head of said bone screw inside a screw hole and preventing retraction of said bone screw from said vertebrae.

20. The locking element of claim 17, wherein said medial and said lateral surfaces of said locking element are adapted to have similar geometry to said bone screws.
